(19) United States
(12) Patent Application Publication
(10) Pub. No.: US 2023/0377697 A1
(43) Pub. Date: Nov. 23, 2023

(54) SYSTEM AND A WAY TO AUTOMATICALLY MONITOR CLINICAL TRIALS - VIRTUAL MONITOR (VM) AND A WAY TO RECORD MEDICAL HISTORY

(71) Applicant: VIRTUAL MONITOR SYSTEMS SPOLKA Z OGRANICZONA ODPOWIEDZIALNOSCIA, Warsaw (PL)

(72) Inventors: Lukasz OLEK, Jozefow (PL); Adam KRUSZEWSKI, Warszawa (PL)

(21) Appl. No.: 18/248,382

(22) PCT Filed: Oct. 12, 2021
(86) PCT No.: PCT/IB21/59339

§ 371 Date: Apr. 9, 2023

(30) Foreign Application Priority Data
Oct. 12, 2020 (PL) 435642

(51) Int. Cl. G16H 10/20; G16H 70/60; G16H 10/60; G06F 40/186; G06F 40/174; G06F 40/58

(52) U.S. Cl. CPC G16H 10/20; G16H 70/60; G16H 10/60; G06F 40/186; G06F 40/174; G06F 40/58; G16H 15/00

(57) ABSTRACT

The clinical trial automatic monitoring system includes a programmable central unit, at least one peripheral device, a server, a database, a network connecting the database to the server and the central unit. The database includes an electronic health records (EHR) module including an interface and source data of one or more patients in the form of an electronic health record. The EHR module is networked to a virtual monitor (VM) having a programmable central processing unit equipped with a microprocessor and memory and networked to a server. The VM includes a case report form (CRF) module, a patient data access module, programmable instructions, a translation engine, a user interface, and a data processing module. The data from the CRF module is analyzed for consistency with the data from the EHR module.

### CROSS-REFERENCE TO RELATED APPLICATIONS

See also Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The subject-matter of the invention is a system and method to automatically monitor clinical trials and a method to record medical history.

### 2. Description of Related Art Including Information Disclosed Under 37 CFR 1.97 and 37 CFR 1.98.

From the state of the art are known ways, devices and systems for facilitating the management of conducting clinical trials.

From patent PL376531 A1, a clinical trial management system is known that includes a client web application, a server and a database. The server provides a number of different applications that users can run, depending on their role in the clinical trial process. Information from the database records about a patient is reported in a manner that depends on the role the user requesting the information plays in the clinical trial process. The document also discloses a method to schedule and track meetings and a method to monitor events in a clinical trial management system that includes conducting an event in a clinical trial protocol; checking the event against business logic rules, industry regulations, and industry standards; and alerting at least one stakeholder of the event.

From patent WO2019143590 A1, a method for identifying errors in clinical trial data and directing workflow in a clinical trial process is known based on a defined clinical trial protocol and risk assessment. The disclosed method includes obtaining clinical trial data from one or more remote entities; generating analytical data by applying one or more algorithms; identifying one or more errors in the clinical trial process by locating one or more deviations in the analytical data; and providing feedback directing the workflow of at least clinical trial personnel or participants based on the generated analytical data.

From patent WO2018164768 A1, a method of analyzing clinical data from a clinical trial in a computer system is known that includes importing the clinical data into a memory device associated with the computer system; storing the clinical data in said memory device, with the microprocessor of the computer system calculating a metric value for the attribute under study by applying the metric function to measurements of a patient variable collected in a plurality of patient visits to a clinical unit; creating, in the memory device, an object of the analytic data, wherein the object of analytic data stores a plurality of metric values, with the microprocessor determining, for each “f‘ metric function and each “v” variable, a “Sf, v (u)” risk score associated with each “u” clinical unit; having an object of risk data created in the memory device, with the risk data object storing the “Sf, v (u)” risk scores for all “f” metric functions, all “v” variables, and all “u” units, with the graphical representation of the risk scares being displayed on a display device associated with the computer system.

Patent EP3241176 A1 discloses a system for identifying and tracking a therapeutic protocol based on cytopathological and genetic data whereby the disclosed system includes a database storing the clinical data, a network connecting the database to an electromagnetic navigation system (EMN) or computing device; a display able to communicate with the computing device; and a user interface presenting, on the display, the patient data including one or more of the target imaging data, target cytopathology, target genetic information, and treatment options, wherein the treatment options are based on a correlation of similarities in the data from a plurality of prior patients and the data associated with the current patient's target.

In contrast, document EP 1940285 B1 discloses a device and a computer-assisted method for analyzing the therapeutic effect of treating patients in a clinical setting whereby the said method includes the following steps: (i) identifying a plurality of patient records in a clinical database, whereby the said records include patient measurements relating to the tests performed during the off-treatment and during the treatment periods; (ii) identifying at least one test in the said plurality of records concerning the k measurements of each patient during the post-treatment period; (iii) identifying at least one test in said plurality of records concerning the measurements of the j factor of each patient during the treatment period; (iv) selecting by way of identifying the best measurement from the said measurements of the k factor of each patient during the said post-treatment period; (v) performing an evaluation of the statistical distribution based on said plurality of records to identify the probability of occurrence of a plurality of the subsets of said measurements during the treatment in excess of such best measurement whereby the evaluation of the statistical distribution is based on a computer model based on random number generation or is derived from a range disparity distribution; (vi) selecting a threshold probability corresponding to the probability of the measurements of the j factor in excess of said best measurement wherein such number of measurements exceeding said best measurement indicates improved performance and thus patient's response to the treatment; (vii) identifying one or more acceptable subsets based on a plurality of measurement subsets during treatment with the respective probabilities which do not exceed the threshold probability; (viii) comparing said measurements with said best measurement during the treatment; and (ix) selecting patients having, as one of the acceptable subsets, a number of measurements of the j factor that exceed the said best measurement.

Based on patent EP3588507 A1, a device and method is known that allows remote monitoring of measurements in clinical trials whereby said method consists of accessing, via a processor, one or more data units of the participant in a clinical trial, which are received over a network connection from a client device configured to perform the clinical trial log application in order to collect the trial [data] which is connected to at least one measurement device whereby, in response to [a signal from] processor detecting that in the unit comprising the data of a participant in the clinical trial one or more corresponding collected clinical trial measurement samples are missing, the processor searches out the connection log of the client device in order to make an entry in the log concerning the problem. Then, after the finding of the entry concerning the connectivity problem in the connectivity log, the processor determines the existence of a potential connectivity problem and corrects the connectivity whereby the correcting of the connectivity includes at least either of: sending an alert to the support unit regarding the potential connectivity problem or sending instructions to the client device to resolve the potential connectivity problem.

The state of the art lacks comprehensive solutions combining the remote monitoring, the data security and the unification of the clinical records.

### BRIEF SUMMARY OF THE INVENTION

The subject-matter of the invention is to provide a new method and system for remote monitoring of clinical trials by forcing the Investigator to describe visits in a manner consistent with the clinical trial protocol and identical to the CRF, and to build mechanisms for automatic transcription of data from the medical reports (MR) to the CRF, and subsequent consistency checks between the CRF and records in the EHR.

The subject-matter of the invention is a clinical trial automatic monitoring system comprising a programmable central unit consisting of a microprocessor and memory, at least one peripheral device, a server, a database, a network connecting the database to the server and the central unit, characterized in that the database is an EUR module comprising an interface and source data of one or more patients in the form of an electronic health record comprising at least personal data, medical history, medications taken; whereby the EHR module is networked to a VM which comprises a programmable central processing unit equipped with a microprocessor and memory and networked to a server, whereby the VM includes:

(a) A CRF module allowing creating clinical record files, equipped with the MR form templates and containing a repository of the stored clinical trial records comprising the data entered during the clinical trial, in particular clinical trial setup, particulars of the staff members conducting the clinical trial, particulars of the patients participating in the clinical trial, observational data concerning one or more patients [enrolled] in the clinical trial, the CRF structures, multilingual MR templates, study results, and clinical analysis data;

(b) A patient data access module connecting to the EHR module interface and importing data from the hospital system to the VM;

(c) Programmable instructions taking the form of an algorithm to generate description of illness based on the clinical trial configuration contained in the clinical trial protocol and an engine to generate MR text form templates with tree structures of fields, with each field containing a value needed to analyze the results of a given clinical trial, and each branch being a node that is assigned a specific text template;

(d) A translation engine containing an algorithm and a repository of medical terms and disease classification codes;

(e) A user interface to present the data for the entry being generated for the CRF form on the display of the central unit and/or the display of peripheral device;

(f) A data processing module equipped with an algorithm, to analyze consistency of the data for the CRF form with the data of the EHR module.

Advantageously, at least one peripheral device is one of the following: a PC, a smartphone, a tablet or a combination thereof.

Advantageously, access to the patient's data is executed using means allowing connecting with the interface of the EHR module, i.e. access via an application programming interface (API) or direct access via the API database.

Advantageously, the system comprises means to verify user credentials consisting of either of the following: a user name and password to access the system, a fingerprint reader, a retinal scanner and/or a magnetic card reader.

Another essential feature of the invention is a method to automatically monitor a clinical trial, characterized in that it comprises the following steps:

(a) The VM monitor interfacing with the EHR module interface and identifying one or more MRs for at least one patient;

(b) Assisting the investigator by providing an interactive MR template in his/her native language, with the option of filling in the blanks with data from the medical study; where, in the event where data that need to be entered based on other already implemented studies, the system, via the patient access module, reads the most recent results in the system and enters them into the template;
(i) generating form fields for additional investigator comments
(ii) validating and saving the template
(iii)creating automatically records in the CRF database containing the data from all the fields completed by the investigator;
(iv) copying and entering the resulting description into the MR in the EHR system;

Another essential feature of the invention is a method for recording the medical history characterized in that it comprises the following steps:

(a) VM connect to the EHR module interface and identify one or more medical history records of at least one patient;

(b) Importing a given patient's test and consultation results from the EHR module into a blank MR form with the tree structure of the fields;

(c) Supporting the investigator's entry of clinical trial data obtained during the follow-up visit by having the engine generate predefined phrase translations corresponding to the characteristics of the field in the investigator's national language;

(d) Generating a completed MR form to describe specific visit and creating a record containing the data from the MR form in the CRF;

(e) Import the completed CRF form into the EHR module;

Advantageously, the method comprises the (f) step whereby the records from the EHR module are automatically reconciled with the data stored in the CRF, which subsequently involves: connecting the patient data access module to a second database of the hospital system, retrieving all stored CRF records available for one or more patients participating in the monitored clinical trial, and searching each field of each form with a search algorithm deep into the tree structure of each form and confirming whether the values stored therein match the data from the EHR module, where if discrepancies are found, the disputed fields are flagged for clarification and a notification of the discrepancies found is sent to the Investigator's peripheral device.

The invention provides the following advantages; it:

Enables monitoring of a clinical trial in an automated manner,

Facilitates describing visits related to clinical trial;

Forces the Investigator to perform actions in conformity with a specific MR template in pace with filling them out, thanks to which he/she can see what data and in what format should be entered;

Allows aggregation and unification of a large pool of clinical data;

Enables working using the national language of the Investigator and at the same time offers a translation engine from/into English;

Provides a uniform form of conducting visit descriptions in clinical trials eliminating differences resulting from the way different investigators work;

Ensures enforced and automatic identity of data needed for a clinical trial and a patient's medical records;

Offers a double-checking functionality—i.e. in the event where data is received the parameters of which do not fall within the pre-defined limits (e.g. the body temperature goes beyond the preset limits) or input data is inconsistent with the fields in the dictionary, then the user becomes alerted about the data which allows him/her to correct the submitted data or to accept and justify it in a separate file available to the Sponsor of the trial, and, possibly, the CRO. However, user's approval is required whenever data goes beyond the pre-defined limits. The user (investigator, clinician) may retain the data after approving them;

It automatically translates these data from the native languages in which the records are kept, i.e., it captures the terms to be transcribed and translates them into English by writing them to the CRF. This means that regardless of the local language of the patient MR files, the CRF database will be populated in English.

It allows research to continue when it is difficult for outsiders to access the hospital, such as during an outbreak of epidemic.

In the system, information appears in the real time.

Allows remote monitoring of a clinical trial without the need to process patients' personal data (patients' personal data in the MR sheet are anonymized).

Prevents errors by way of monitoring whether entries in CRF and EHR are identical.

Ensures security of the entered data. After the physician completes an entry and sends the data to the database, nothing can be changed in the patient's medical history.

### BRIEF DESCRIPTION OF THE SEVERAL VIEWS OF THE DRAWINGS

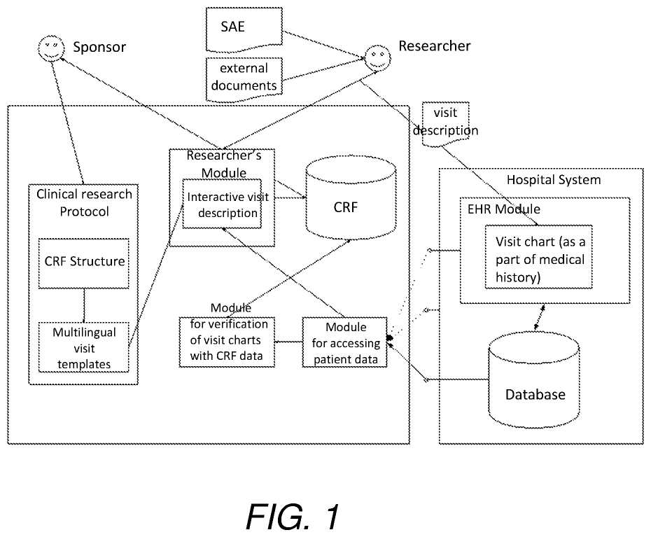

The invention is shown in embodiments in figures and in the illustration where FIG. 1 schematically shows a system in accordance with the invention.

### DETAILED DESCRIPTION OF THE INVENTION

### EXAMPLE 1

A system for automatic monitoring of clinical trials in accordance with the invention is schematically shown in FIG. 1.

The system according to the invention includes a programmable central unit comprising a microprocessor and memory, a server, first and second databases, a network connecting the database to the server, and a central unit comprising a microprocessor and memory (e.g., a desktop computer). Further, the system includes at least one peripheral device that allows the Investigator to remotely access the system. The number of peripherals depends on the number of investigators (e.g., physicians, scientists, medical staff) or other entities (e.g., sponsors, patients, network administrators) participating in and/or overseeing the clinical trial.

At the same time, for each of the mentioned entities, access to the data stored and processed in the system conforming to the invention will depend on the level of privileges with various security features to verify the privileges, e.g. a pre-set user password, verification of a retinal or fingerprint scan, privileges encoded on a magnetic card, etc.

On the other hand, the peripheral device in accordance with the invention may be either a smartphone, a computer (PC, laptop) or a tablet.

As described above, the system according to the invention includes a CRF database and an EHR database. The EHR module comprises of an interface and the source data for one or more patients in the form of an electronic MR including at least the personal information, medical history, medications taken. It is an external module, provided through, for example, an application programming interface (API) or direct access to the database, e.g. in the form of the appropriate views. The EHR module is networked to a monitor VM.

Said VM comprises a programmable central unit equipped with a microprocessor and memory and networked to a server, which provides the VM functionality over the network, where, as indicated in FIG. 1, the VM includes:

A CRF module to create clinical trial record files that is equipped with MR form templates and contains a repository of the clinical trial records (including: CRF structures, multilingual MR templates, study results, clinical analysis data);

The database (i.e., the CRF module repository) stores the data entered during the clinical trial, in particular clinical trial setup, clinical trial staffs data, clinical trial patients' data, observational data on one or more patients in the clinical trial, CRF structures, multilingual patient's visit templates, study results, data from clinical analyses;

Patient data access module that connects to the EHR module interface and imports data from the hospital system into the VM virtual monitor;

Programmable instructions in the form of an algorithm to generate a disease description based on the clinical trial configuration included in the clinical trial protocol and a MR text template engine with a tree structure of the fields, each field containing a value needed to analyze the results of a given clinical trial, and each branch being a node that is assigned a specific text template;

A translation engine containing an algorithm and a repository of medical terms and disease classification codes;

A user interface to present the data for the entry being generated for the CRF form on the display of the central unit and/or the display of peripheral device;

A data processing module equipped with an algorithm, to analyze consistency of the data for the CRF form with the data of the EHR module.

### EXAMPLE 2

Exemplary implementation of the MR template generating mechanism

The CRF form has fields with a tree structure divided into: visits, pages, and sections/single fields. Each field ultimately contains a certain value required for analysis of the results of the trial, sometimes the values in the fields are optional and sometimes they are required conditionally (depending on the values of other fields).

Let's consider an exemplary CRF system with the following field structure:

CRF System

```
1) Visit 1
  a) Page_A
    i) Section I
      (1) Field_1
      (2) Field_2
    it) Section_II
      (1) Field_1
      (2) Field_2
2) Visit 2
  a) ...
```

The template, assigned to selected nodes of the CRF form, would consist of text containing certain commands, such as.:

The curly brackets would contain the name of the variable, e.g.: “Patient has pressure {X}”, where X is a reference to a specific field in the CRF.

We could use the if-else clauses to define conditional descriptions: {“% if condition %} Text if condition is true {% else %} Text if condition is false {% endif %}”

We would use the existing template engine to implement the language of the templates. The exact syntax would result from the chosen language.

The template language engine would be used to create an interactive user interface for the Investigator, who, using such interface, will select specific patient and specific visit number. The system will show him visit description template allowing filling in the values of the variables values. When a specific variable is selected, e.g., field X in sentence: “Patient has blood pressure {X},” the system launches an editor depending on the type of variable X:
for the numeric X variables, the system will limit the input characters to digits and the fractional part separator;
for the text X variables, the system will allow entering any value;
for the single- or multiple-choice X variables, the system will display a list of values to choose from a list of pre-defined values.

Additionally, additional conditions may be set for the X variables, such as e.g. the range of numeric values. When a value being entered does not meet the conditions, the system will immediately display a warning.

Once the value of any variable is entered, the system checks if the template itself has not been modified (e.g. because of the possibility that conditional sections might exist in the MR template) and updates the description accordingly.

The system can use the mechanism of translating the templates into different national languages of the countries where the trial is conducted. To this end, it is possible to use readymade internationalization software packages such as gettext, where for an English phrase e.g.: “Patient has blood pressure {X}.”, you can specify Polish version: “Cisnienie krwi pacjenta wynosi {X}.” In addition, the values for the single- or multiple-choice variables would also be entered in the dictionaries so as to allow their translation.

With the package as described above, the physician could describe each event (e.g., patient parameter) in the MR chart in a uniform pre-defined way yielding uniformity of all entries in the MR chart, EHR, and CRF.

Each template would be subordinated to a specific node in the CRF. In the template description, we could refer to any field pre-defined in the CRF in much the same way we address folders on a computer, e.g., when defining a template for the node “Visit_I/Page_A/Section_I” we might refer to the following fields:

{Field_I}—to a specific field in section Section_I

{ . . . /Section_2/Field_I}—to the specified field in the adjacent section in a relative way:
double dot—means a node above the node for which we define
o/—indicates selection of a specific “sub-node”

{Visit_I/Page_B/Field_2}—starting the variable description with means an absolute addressing, starting from the beginning of the CRF structure.

The algorithm would work like this while in-depth searching through the CRF tree structure, and after the algorithm finds a template assigned to a specific node, it will add the generated string at the end of the visit description, while inserting an interactive value editor in place of the variables.

The Investigator's user interface will display the template with the ability to change the values of the template variables. The very content of the sentences provided in the template mechanism would be non-editable, while the Investigator could supplement the description with additional sentences, between the sections generated from the template.

### Protocol Violation

In clinical trials, there is a need to describe situations in which the Investigator has acted inconsistently with the established clinical trial protocol. In these situations, the Investigator would be expected to complete an additional field in the CRF and include this information in the patient MR in the EHR.

Such situations would be implemented in the VM by providing “protocol violation” fields in specific locations in the CRF, which would be visible in the MR template depending on a number of conditions checking compliance of the data with the clinical trial protocol (based on the condition mechanism embedded in the templates assigned to CRF fields). The field would only show up if the premises were met (the protocol was violated).

Multi-dimensional data (e.g. Serious Adverse Events (SAE))

In certain situations, the tree structure of a field as described so far is insufficient because for example the data that should be entered in the CRF have the form of a table (i.e. are two-dimensional), or a nested table (are more-than-two-dimensional). An example are Serious Adverse Events, where the physician should provide a description of each event with the additional attributes (most importantly, whether the event was related to the received medication). Due to the fact that most of the EHR systems only allow entering text data in the description in the MR sheet, we propose a solution of transforming multidimensional data structures into a text list, so that the Investigator can define them in this form and save them to the description in the MR sheet.

Whenever another data dimension is needed, we would add a list in the MR template using command: {% list_list name %} ended with command: {% endlist %}. Between these commands, definition of a single element of the list (as shown earlier) appears. Additionally, as part of the description of an element of the list we can use the {% index list_name %} command, which, in the description of the visit, will become transformed into a list index (the number of an item listed in the list).

An example MR template may look as follows:

CRF

| | |
|---|---|
| Description | Severity |
| of Events | |

Visit template

```
The following adverse events
occurred:
(% list sae%)
    Event {%index sae%}:
  (Event Description)
  (severity: (Severity))
(% endlist %)
```

A MR template prepared in this way will be displayed to the Investigator as a list, where he/she can add items by clicking the appropriate button. In the event when a list contains no items, word: “none” will appear in the description of the visit:

Interactive Description of Events

```
The following adverse events
occurred:
  - None
  - [symbol]
```

After clicking the “plus” button, the Investigator will see a new item of the list in conformity with the structure defined in the MR template, where specific fields can be edited:

Interactive Description of Events

```
The following adverse events
occurred:
  - Event 1: Headache right after
    getting out of bed
    (severity: mild)
  - [symbol]
```

The investigator will be able to enter an opinion on the adverse events (SAEs) by indicating whether they believe the SAE was related to the study drug.

### EXAMPLE 3

In this non-restrictive example of implementation, recording the medical history in the system in accordance with the invention comprises the following steps:

(a) VM connect to the EHR module interface and identify one or more medical history records of at least one patient;

(b) Importing a given patient's test and consultation results from the EHR module into a blank MR form with the tree structure of the fields;

(c) Supporting the investigator's entry of clinical trial data obtained during the follow-up visit by having the engine generate predefined phrase translations corresponding to the characteristics of the field in the investigator's national language;

(d) Generating a completed MR form describing a given visit and creating a record in CRF containing data from the MR form;

(e) Import the completed CRF form into the EHR module;

Next, the records from the EHR module are being automatically reconciled with the data stored in the CRF, which includes successively: the module of access to patients* data connecting with another database in the hospital's system, retrieving all the CRF records stored therein which are available for one or more patients participating in the monitored clinical trial, and searching, employing a search algorithm through each and every field of each form deep into the tree structure of each form to confirm whether the data stored therein are consistent with the data from the EHR module; in the event that discrepancies are found, the discrepant fields are marked for clarification and a notification about the discrepancies found is sent to the investigator's peripheral device.

1. A system for automatic monitoring of clinical trials, comprising:
a programmable central unit being comprised of:
a microprocessor and memory,
at least one peripheral device,
a server,
a database, and
a network connecting the database to the server and the central unit,
wherein the database is an electronic health records (EHR) module comprising an interface and source data of one or more patients in the form of an electronic health record comprising at least the personal data, medical history, medication taken; and
a virtual monitor (VM) being networked to the EHR module and being comprised of:
a programmable central unit equipped with a microprocessor and memory and networked to a server.
a case report form (CRF) module allowing creating clinical record files, equipped with the medical records (MR) form templates and containing a repository of the stared clinical trial records comprising the data entered during the clinical trial, in particular: clinical trial setup, particulars of the staff members conducting the clinical trial, particulars of the patients participating in the clinical trial, observational data concerning one or more patients [enrolled] in the clinical trial, the CRF structures, multilingual MR templates, study results, and clinical analysis data,
a patient data access module connecting to the EHR module interface and importing data from the hospital system to the VM,
programmable instructions taking the form of an algorithm to generate description of illness based on the clinical trial configuration contained in the clinical trial protocol and an engine to generate MR text form templates with tree structures of fields, with each field containing a value needed to analyze the results of a given clinical trial, and each branch being a node that is assigned a specific text template,
a translation engine containing an algorithm and a repository of medical terms and disease classification codes,
a user interface to present the data for the entry being generated for the CRF form on the display of the central unit and/or the display of peripheral device, and
a data processing module equipped with an algorithm, to analyze consistency of the data for the CRF form with the data of the EHR module.

2. The system, according to claim 1, wherein the at least one peripheral device is selected from the following: a PC, a smartphone, a tablet or a combination thereof.

3. The system, according to claim 1, wherein the patient data access module comprises means allowing it to connect with the interface of the EUR module, i.e. access via an application programming interface (API) or direct access via the API database.

4. The system, according to claim 1, further comprising: a means to verify user credentials consisting of either of the following:
a user name and password to access the system, a fingerprint reader, a retinal scanner and/or a magnetic card reader.

5. A method to automatically monitor a clinical trial, the method comprising the steps of:
(a) The VM monitor interfacing with the EHR module interface and identifying one or more MRs for at least one patient; and
(b) Assisting the investigator by providing an interactive MR template in his/her native language, with the option of filling in the blanks with data from the medical study; where, in the event where data that need to be entered based on other already implemented studies, the system, via the patient access module, reads the most recent results in the system and enters them into the template;
(i) generating form fields for additional investigator comments;
(ii) validating and saving the template;
(iii)creating automatically records in the CRF database containing the data from all the fields completed by the investigator;
(iv) copying and entering the resulting description into the MR in the EHR system.

6. A method of recording medical history, the method comprising the steps of:
(a) VM connecting to the EHR module interface and identify one or more medical history records of at least one patient;
(b) Importing a given patient's test and consultation results from the EHR module into a blank MR form with the tree structure of the fields;
(c) Supporting the investigator's entry of clinical trial data obtained during the follow-up visit by having the engine generate predefined phrase translations corresponding to the characteristics of the field in the investigator's national language;
(d) Generating a completed MR form describing a given visit and creating a record in CRF containing data from the MR form; and
(e) Import the completed CRF form into the EHR module.

7. The method according to claim 6, wherein step (f) further comprises: automatically reconciling the records from the EHR module with the data stored in the CRF, which subsequently involves: connecting the patient data access module to a second database of the hospital system, retrieving all stored CRF records available for one or more patients participating in the monitored clinical trial, and searching each field of each form with a search algorithm deep into the tree structure of each form and confirming whether the values stored therein match the data from the EHR module, where if discrepancies are found, the disputed fields are flagged for clarification and a notification of the discrepancies found is sent to the Investigator's peripheral device.
